(19) United States
(12) Patent Application Publication
(10) Pub. No.: US 2017/0189710 A1
(43) Pub. Date: Jul. 6, 2017

(54) DEVICE AND METHOD FOR LOW-NOISE MAGNETIC NEUROSTIMULATION

(71) Applicants: Stefan M Goetz, Durham, NC (US); David L.K. Murphy, Durham, NC (US); Angel V. Peterchev, Durham, NC (US)

(72) Inventors: Stefan M Goetz, Durham, NC (US); David L.K. Murphy, Durham, NC (US); Angel V. Peterchev, Durham, NC (US)

(21) Appl. No.: 15/320,023

(22) PCT Filed: Jun. 20, 2015
(86) PCT No.: PCT/EP2015/063889

§ 371 Date: Feb. 16, 2017

(30) Foreign Application Priority Data
Jun. 20, 2014 (DE) 10 2014 008 820.0
Jun. 20, 2014 (DE) 20 2014 004 930.0
Jun. 20, 2014 (DE) 20 2014 004 932.7

(51) Int. Cl. A61N 2/02; H01F 27/32; A61N 2/00; A61N 2/12; A61N 1/06; A61N 1/40

(52) U.S. Cl. CPC A61N 2/02; A61N 1/06; A61N 1/40; A61N 2/006; A61N 2/12; H01F 27/327; A61N 2/004

(57) ABSTRACT

The present invention relates to a device and a method for the stimulation of nerve and muscle cells according to the principle of magnetic stimulation, wherein the invention has a significantly reduced sound emission for the same stimulus intensity when compared to the prior art. The sound emission in the form of a click noise, which, in the magnetic stimulation, determines on the one hand an important safety risk and on the other hand causes an undesired, uncontrollable sensory-auditory brain stimulation is reduced in the present invention by increasing the frequency of a substantial portion of the spectrum of the pulse, preferably up to or beyond the human hearing range. The invention further relates to a quieter coil technology which reduces the conversion of electrical energy into mechanical-acoustic oscillations, prevents the transfer of said oscillations to the surface by resilient decoupling and instead converts the mechanical-acoustic energy via viscoelastic deformation of the material into heat.

### INTRODUCTION AND PRIOR ART

The present invention relates to a device and a method for the stimulation of neurons and muscle cells according to the principle of magnetic stimulation, the invention generating substantially less acoustic sound emission for the same stimulus intensity compared to the prior art. The present invention reduces the acoustic sound emission in the form of a clicking sound, which, in magnetic stimulation, firstly is an important safety risk and secondly causes undesired uncontrollable sensory-auditory brain stimulation, by increasing the frequency of a substantial portion of the spectrum of the pulse, preferably to or above the human hearing range.

Further, the present invention relates to a quieter coil technology which reduces the conversion of electrical energy into mechanic-acoustic oscillations, suppresses the transmission thereof to the surface by elastic decoupling and instead converts the mechanic-acoustic energy into heat by viscoelastic material deformation.

### Significance

Transcranial magnetic stimulation (TMS) is a method for non-invasive brain stimulation with brief, strong magnetic pulses which induce an electric field in the brain. This technique is widely used in the neurosciences, in particular, as a method for probing specific brain functions. Further, said technique is approved by, inter alia, the US regulators FDA for clinical treatment of depression, and is under study for a number of other psychiatric and neurological disorders and syndromes. Moreover, TMS has been demonstrated to make it possible to temporarily enhance individual cognitive functions in healthy subjects.

A TMS machine includes a pulse source or pulse generator and a stimulation coil which is placed on the subject's head. Typical TMS machines generate coil current pulses in the coil, said coil current pulses being sinusoidal with a dominant main frequency (which is also the fundamental frequency in this case) of about 1-5 kHz in the case of currents up to 8 kA, and magnetic field strengths at the coil surface of up to 2.5 T. The high amplitude pulses result in mechanical forces, caused by electromagnetic effects, within the pulse generator, the coil, and the cable connecting these, which in turn entails loud sounds. Of these, the sound of the coil is dominant due to the strong magnetic field in the coil. Further, the sound of the coil is the most difficult to suppress since the coil is placed on the subject's head, from where the acoustic sound is conducted by air and the skull bone [Nikouline V., Ruohonen J., and Ilmoniemi R. J. (1999). The role of the coil click in TMS assessed with simultaneous EEG. Clinical Neurophysiology, 110(8):1325-1328.]. The loud clicking sound on account of the strong forces may reach an acoustic sound pressure level of 120-140 dB at a distance of 10-20 cm and has a spectral peak power in the 1-7 kHz range [Starck J., Rimpiläinen I., Pyykkö I, and Toppila E. (1996). The noise level in magnetic stimulation. Scandinavian Audiology, 25(4): 223-226; Counter S. A., Borg E. (1992) Analysis of the coil generated impulse noise in extracranial magnetic stimulation. Electroencephalography and Clinical Neurophysiology, 85(4):280-288.]. The loud sound generated by conventional machines is a significant limitation of TMS technology, having the following key problems:

(1) The loud click sound may cause hearing damage in the TMS subject, TMS operator, and other persons or experimental animals in the vicinity of the system [Counter S. A., Borg E. (1992) Analysis of the coil generated impulse noise in extracranial magnetic stimulation. Electroencephalography and Clinical Neurophysiology, 85(4):280-288; Counter S. A., Borg E., and Lofqvist L. (1991). Acoustic trauma in extracranial magnetic brain stimulation. Electroencephalography and Clinical Neurophysiology, 78(3):173-184; Rossi S., Hallett M., Rossini P. M., and Pascual-Leone A. (2009). Safety, ethical considerations, and application guidelines for the use of transcranial magnetic stimulation in clinical practice and research. Clinical Neurophysiology, 120(12):2008-2039.]. It is for this reason that anyone in the immediate vicinity of a TMS machine should wear hearing protection, for example earplugs or earphones [Rossi S., Hallett M., Rossini P. M., and Pascual-Leone A. (2009). Safety, ethical considerations, and application guidelines for the use of transcranial magnetic stimulation in clinical practice and research. Clinical Neurophysiology, 120(12):2008-2039.]. Failure of the hearing protection harbors the risk of hearing loss, as exemplified by the occurrence of permanent hearing loss in a subject whose earplug appears to have fallen out during an rTMS session [Zangen, A., Y. Roth, et al. (2005). Transcranial magnetic stimulation of deep brain regions: evidence for efficacy of the H-coil. Clinical Neurophysiology, 116(4):775-779.]. The risk of hearing loss may be higher in children [Rossi S., Hallett M., Rossini P. M., and Pascual-Leone A. (2009). Safety, ethical considerations, and application guidelines for the use of transcranial magnetic stimulation in clinical practice and research. Clinical Neurophysiology, 120(12):2008-2039.]. The risk is further increased in environments where the mechanical forces are increased and/or acoustic feedback or reflections are/is present, for example in magnetic resonance imaging (MRI) scanners during interleaved TMS and functional imaging.

(2) Even with hearing protection, the auditory perception of the TMS sound is substantial and often unpleasant or intolerable to the subject or patient, the operator, or other persons in the vicinity of the TMS machine. Intolerance may be particularly pronounced for persons with increased sensitivity to noise (hyperacusis). Hyperacusis is estimated to affect 8-15% of the general population [Baguley, D. M. (2003). Hyperacusis. Journal of the Royal Society of Medicine, 96(12): 582-585; Coelho C. B., Sanchez T. G., and Tyler R. S. (2007). Hyperacusis, sound annoyance, and loudness hypersensitivity in children. Progress in brain research 166:169-178.] and has a higher prevalence in patients with some psychiatric and neurological disorders, including tinnitus, migraine, autism spectrum disorders, depression, post-traumatic stress disorders and other anxiety disorders. For these disorders, TMS is either approved (depression) or investigated as a therapeutic intervention. Aside from that, tension-type headache is the most common side effect of rTMS, occurring approximately in 23%-58% of subjects or patients and in 16%-55% of the control group [Loo C. K., McFarcluhar T. F., and Mitchell P. B. (2008). A review of the safety of repetitive transcranial magnetic stimulation as a clinical treatment for depression. International Journal of Neuropsychopharmacology, 11(1):131-147; Machii K., Cohen D., Ramos-Estebanez C., and Pascual-Leone A. (2006). Safety of rTMS to non-motor cortical areas in healthy participants and patients. Clinical Neurophysiology, 117(2):455-471; Janicak P. G., O'Reardon J. P., Sampson S. M., Husain M. M., Lisanby S. H., Rado T. J., Heart K. L., and Demitrack M. A. (2008). Transcranial magnetic stimulation in the treatment of major depressive disorder: A comprehensive summary of safety experience from acute exposure, extended exposure, and during reintroduction treatment. Journal of Clinical Psychiatry, 69(2):222-232.]. Since tension-type headache may be triggered by exposure to noise [Martin P. R., Reece J., and Forsyth M. (2006). Noise as a trigger for headaches: Relationship between exposure and sensitivity. Headache, 46(6):962-972; Wöber C. and Wöber-Bingol C. (2010). Triggers of migraine and tension-type headache. Handbook of Clinical Neurology, 97:161-172.], it is a distinct possibility that the noise generated by the TMS machine is an important contributor. Therefore, in some patient groups, the TMS noise may present an obstacle to convalescence brought about by the treatment.

(3) The auditory perception of the TMS sound results in evoked responses in the brain which are not generated by the magnetic stimulus, but are nevertheless synchronous therewith. Thus, it is difficult to separate the effect of the magnetic pulse from the auditory response [Komssi S., and Kahkonen S. (2006). The novelty value of the combined use of electroencephalography and transcranial magnetic stimulation for neuroscience research. Brain Research Reviews, 52(1):183-192.]. This may impede experimental studies and bring about unintended neuromodulation or interaction between acoustic sound stimulus and electromagnetic stimulus in clinical applications. Repetitive auditory stimulation, for instance, may also induce long term potentiation (LTP) in the brain [Clapp W. C., Kirk I. J., Hamm J. P., Shepherd D., and Teyler T. J. (2005). Induction of LTP in the human auditory cortex by sensory stimulation. European Journal of Neuroscience, 22(5):1135-1140; Clapp W. C., Hamm J. P., Kirk I. J., and Teyler T. J. (2012). Translating Long-Term Potentiation from Animals to Humans: A Novel Method for Noninvasive Assessment of Cortical Plasticity. Biological Psychiatry, 71(6):496-502; Zaehle T., Clapp W. C., Hamm J. P., Meyer M., and Kirk I. J. (2007). Induction of LTP-like changes in human auditory cortex by rapid auditory stimulation: An FMRI study. Restorative Neurology and Neuroscience, 25 (3-4):251-259.], which overlays the modulation effect in rTMS. By way of example, one of the rTMS depression treatments, which the FDA has approved in the US, uses 10 Hz pulse trains. This corresponds to the repetition rate of highest auditory cortex sensitivity (10-14 Hz) and is very close to 13 Hz, the frequency at which auditory-induced LTP has been demonstrated in humans [Clapp W. C., Hamm J. P., Kirk I. J., and Teyler T. J. (2012). Translating Long-Term Potentiation from Animals to Humans: A Novel Method for Noninvasive Assessment of Cortical Plasticity. Biological Psychiatry, 71(6):496-502.].

(4) The loud clicking sounds of TMS machines presents a challenge to the environment where the TMS machine is assembled and operated. Since the sound of TMS machines may penetrate neighboring rooms in the building, researchers and physicians using TMS machines face challenges from occupants, colleagues and/or the building management. Further, noise emission/noise reception is restricted by regulations in many countries. Since many medical practices are not located in designated industrial areas, noise limits as strict as 55 dB(A) outside and 35 dB(A) in neighboring units within the building may apply [TAL (1998), German Technical Instruction on Noise Protection According to the Federal Control of Pollution Act BImSchG/Technische Anleitung zum Schutz gegen Laerm erlassen auf der Basis des Bundesimmissionsschutzgesetzes. GMBI No. 26/1998, p. 503.]. Without enhanced noise damping measures in the building, the use of TMS for medical purposes may be restricted.

Many of the aforementioned considerations likewise apply to machines for peripheral magnetic stimulation. It is for this reason that the invention may also be applied to peripheral magnetic stimulation machines.

### Solution Approaches According to the Prior Art

To reduce the noise generated by TMS machines, some manufacturers use methods to dampen oscillations in the stimulation coil. The effectiveness of this approach is limited, as evidenced by the high noise level of commercially available machines [Starck J., Rimpiläinen I., Pyykkö I, and Toppila E. (1996). The noise level in magnetic stimulation. Scandinavian Audiology, 25(4): 223-226; Counter S. A., Borg E. (1992) Analysis of the coil generated impulse noise in extracranial magnetic stimulation. Electroencephalography and Clinical Neurophysiology, 85(4):280-288.]. A proposed approach for more drastic noise reduction involves placing the coil winding in an evacuated vessel [Ilmoniemi R. J. et al. (1997). EP 1042032, WO 99/27995]. This approach attempts to the acoustic emission by removing, from the vicinity of the coil winding, all media which could transport the acoustic sound. This approach to the problem, however, has a number of shortcomings: (1) As a rule, the air-tight evacuated vessel around the coil increases the spacing between the coil winding and the stimulation target and therefore worsens the electromagnetic coupling to the target, as well as the electrical efficiency of the system. (2) There still are alternative acoustic sound paths starting from the points where the coil conductor enters the evacuated vessel, starting from the coil cable, and starting from the pulse generator. (3) An evacuated vessel is large, inflexible, impractical, probably fragile, and expensive.

The relatively extreme, but nevertheless not very effective or practical approaches from the prior art show that there is a compelling need for the development of a TMS machine which generates less noise. The present invention comprises a concept of quiet TMS technology, which substantially reduces the noise generated by TMS.

### FIGURES

FIG. 1 shows the capacitor voltage V_{Cth} (101) and the peak coil current I_{Lpk} (102), approximately at the cortical activation threshold as a function of pulse fundamental frequency f₀ assuming a first order neural membrane model with a time constant τₘ=196 μs. The data are based on the parameters of a commercially available Magstim figure-8 coil. The values are plotted for a number of turns, N, equaling 18 (conventional Magstim figure-8 coil) and for N=9.

FIG. 2 shows the amplitude spectrum for the coil current of a conventional biphasic Magstim figure-8 pulse (201, black) and of a 30 kHz biphasic pulse (202, gray) at the stimulus threshold.

FIG. 3 shows two embodiments of acoustically more advantageous current pulse wave forms (301, 302), spga, spgb, within the meaning of the invention. Both pulses are normalized to their individual stimulus threshold for a human motor neuron. In contrast to conventional waveforms in magnetic stimulation, the fundamental frequency or carrier frequency of the electromagnetic oscillation is significantly higher in order to shift a substantial portion of the energy above the hearing range. Further, the oscillations are not abruptly turned on or off at specific points in time, e.g. at zero-current points of the sinusoidal curve with an otherwise approximately constant amplitude, but rather are modulated by a pulse envelope which smooths the start of the pulse (attack) and the end of the pulse (decay) of the waveform. The resulting waveforms are bandwidth-limited and do not exhibit strong sidebands in the spectrum, as depicted in FIG. 4.

FIG. 4 shows the amplitude spectrum of the spga (404) and spgb (405) waveforms from FIG. 3 and compares these to simpler waveforms (“30 kHz×1” (401): biphasic waveform with a carrier frequency of 30 kHz, i.e. a single period of a sine current/cosine voltage and a fundamental frequency of 30 kHz; “30 kHz×2” (402): polyphasic waveform with two periods at a fundamental frequency of 30 kHz; “30 kHz×3” (403): polyphasic waveform with three periods at a fundamental frequency of 30 kHz). All individual curves are normalized with respect to their individual stimulus threshold for comparison purposes. The strong sidebands of the biphasic pulse and of the polyphasic pulse, which reach far into the hearing range, are suppressed for the spga and spgb waveforms. Consequently, the spectral power of the electromagnetic oscillations at lower frequencies, which, in turn, excite the acoustic oscillations that generate the sound emission, is significantly reduced compared to sinusoidal waveforms with similar fundamental frequencies, while the stimulation strength is preserved. The use of waveforms with specific spectral characteristics for controlling the acoustic sound emission of the machine has previously been unknown in the field of magnetic stimulation.

FIG. 5 shows a cross section of a coil in accordance with the first embodiment of the mechanical part of the invention. The individual turns of the conductor (501) are electrically insulated and securely connected to one another by mechanical means. This secure connection (503) is implemented with a high stiffness (characterized by a high Young's modulus). The connection between the outermost turn and the cable or a second coil ring, for example in a so-called figure-of-eight coil or butterfly coil may be used to form a mechanically stabilizing beam, as indicated in the figure. Further, all gaps may be filled with stiff materials, as indicated in the figure by reference sign (502) (also “stiff core”). The entire stiff block, which is composed of the individual conductors, is from the casing and the surroundings by a layer made of viscoelastic material (504) (high η value, a high Young's modulus is also advantageous) and an additional layer of a highly elastic material (505) (low Young's modulus and Shore hardness). This layer sequence may be repeated. Further, the sequence may also begin and end with a viscoelastic layer, i.e. the latter may form the innermost layer and outermost layer. A casing (506), which is preferably stiff and/or high-mass, seals the coil to the outside and forms the interface to the surroundings (possibly enveloped in damping foam or further materials). Since a specific side is usually applied to the subject, the layer thicknesses may deviate at this side (e.g. be thinner) from those on the other sides. Likewise, the remaining sides need not have equal layer thicknesses amongst themselves.

Conductor (501): preferably with a high density, preferably stiff, preferably not too thin (to avoid bending modes in the transverse direction), preferably no inhomogeneous mass or mass density (similar thickness to avoid a tuning fork effect); stiff core (502); preferably stiff connection (503): for example epoxy-kapton-epoxy compound, fiber composite, glass wool, aramid-epoxy composite (in the case of kapton or polyimides with a surface treatment as adhesion promoter); stiff epoxide or cyanoacrylate epoxide; highly viscoelastic layer (504): preferably a high Young's modulus, preferably a high viscosity, for stiffening and producing mechanical energy losses; highly elastic layer (505): for decoupling; casing (506): preferably stiff and high mass, optional struts for stiffening and softer inserts for controlled production of mechanical modes.

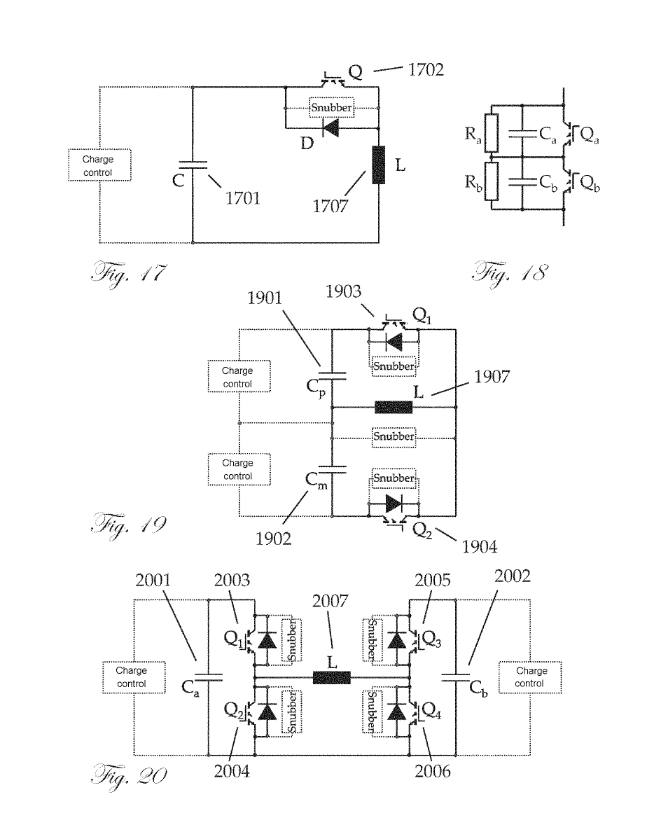

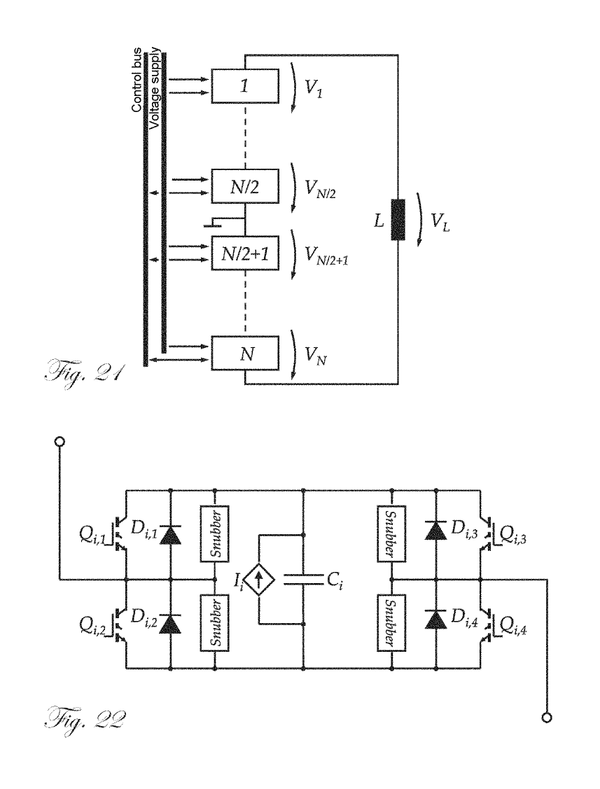

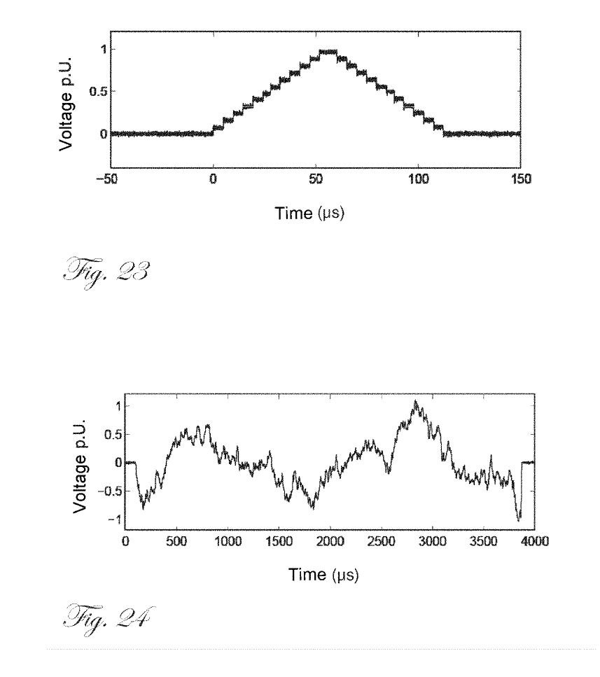

FIG. 6 shows a special embodiment, in which the conductors from the embodiment in FIG. 5 are formed by a copper-clad steel conductor (601). In this special case, the conductor is embodied as a flat strip line with a steel core, which is covered by copper on both sides. Other conductor shapes and cross sections may likewise be used.

FIG. 7 shows a modification of the coil cross section from FIG. 5, in which the effect of the viscoelastic layer is enhanced by an additional stiff layer (708). In this case, the viscoelastic layer (704) comes to rest between two stiff layers (702, 708). This structure forces oscillations to drive the viscous material properties of this layer at all times and cause shear loading, bending and compression; otherwise, the oscillations could merely displace the entire viscoelastic layer, which is relatively stiff, without a (lossy) change in form of the material, or excite modes with a relatively low viscous energy loss. An alternative to an additional stiff layer could be, furthermore, stiff grains or beams in the viscoelastic layer, which force a deformation or bending of the viscoelastic material.

FIG. 8 shows a cross section of a coil in accordance with the second embodiment of the mechanical part of the invention. The individual turns (801) are treated on an individual basis just like the larger block which, in the first embodiment (e.g. FIG. 5) was formed en masse by some or all conductors or turns of the same conductor (801). The individual turns are surrounded by associated viscoelastic layers (803) and associated elastic layers (802). In the case of layer thicknesses which are large compared to the distance between the turns, the viscoelastic layers and/or the elastic layers of individual, e.g. adjacent conductors or turns may contact one another and form a single contiguous layer. Should sufficient space be present, as is depicted here, the remaining gaps and interstices in the coil may be filled by the viscoelastic material (804). Alternatively, the gaps and interstices may be filled with the material of the closest layer in each case. For the present embodiment, the casing encloses the coil and forms the surface to the surroundings (possibly enveloped in damping foam or further materials). The conductors may have a round or oval cross section for a good surface-to-volume ratio. Further, the conductor may be a copper-clad conductor, for example with a steel core. Re (801): conductor, optionally filled with steel; re (802): elastic layer or tube; re (803): viscoelastic layer or tube (high η*E); re (804): cast, optionally with viscoelastic elements, layers or regions.

FIG. 9 shows the surface of a coil which is further strengthened in part using known measures, for example by means of beams, in order to increase the stiffness and/or the mass in general or for specific (mechanical) modes.

FIG. 10 shows a simplified equivalent-circuit model of the acoustic conditions of the second part of the invention and simplifies equivalences using electronic elements. A pressure source (1001), i.e. a mechanical equivalent of an electrical voltage source, on the left-hand side represents the conversion of electromagnetic energy into the acoustic domain. The high stiffness (Eₛₜ and Eₛₗ) (1002) and the high mass (mₛ) (1003) of the conductor, as is preferred by the invention, increases the input impedance and minimizes the amount of converted energy. Damping and decoupling units i, each formed by respectively one viscoelastic layer (with viscosities η_{vl,i} and η_{vt, i}) and one elastic layer (with Young's moduli of E_{el,i} and E_{et,i}), convert the energy into heat and in each case decouple the left-hand side thereof from the right-hand side thereof in the circuit representation. These units may be repeated. The casing with mass m_{c} and Young's moduli E_{cl} and E_{ct} forms the interface to the surroundings, to which it emits sound borne by air and borne by a body. The equivalent electrical elements are an approximation to the greatest possible extent because virtually all known materials have a strong frequency dependence in the parameters thereof and significant nonlinearities. Further, a description in the form of a one-dimensional circuit is only able to approximate the complicated three-dimensional geometric conditions.

The elements are distinguished as pressure source (1001), high stiffness (1002), high mass (1003), high viscosity/viscoelasticity (1004) and high elasticity and low stiffness (1005). The blocks are the source with a high source impedance (1006), damping and/or decoupling (1007) and casing (1008).

FIG. 11 shows equivalencies between electrical and mechanical/acoustic variables.

FIG. 12 illustrates forces which cause oscillations in a coil. Illustration 1201 elucidates the dominant direction of the forces between the conductor turns (1204, 1205, 1206) in a coil, said forces compressing the material between two conductors or conductor turns (1204, 1205, 1206). Illustration 1202 shows the conversion of bending oscillations in the conductor core into a shear load in the viscoelastic layer. Illustration 1203 shows longitudinal oscillations (consequently contraction or translation of the materials), which have more significance for high-frequency frequency components, predominantly above the hearing limit, for TMS coils, depending on the specific material properties.

FIG. 13 shows measured waveforms of a TMS pulse with a period of 300 μs (1301, 1303) and of a shorter pulse with the duration of 45 μs (1302, 1304). Both pulses were produced using a controllable pulse parameter TMS machine (cTMS) and a circular coil. The electric field produced by each pulse was measured using a single-turn dI/dt probe. The peak neuronal depolarization induced by each pulse was modeled by the signal of the probe being conducted through a first-order low-pass filter with a time constant of 150 μs. The intensity of each pulse was selected in such a way that each pulse produces a depolarization (measured peak-to-peak) of 1000 mV. Once matched, the acoustic signal produced by the round coil was recorded using an AKG C214 microphone. Both the microphone and the coil were placed into an acoustically isolated chamber in order to reduce background noise and isolate the coil acoustic sound from the sound generated by the machine during the pulse. A second matched AKG C214 microphone recorded sounds in the chamber such that it was possible to monitor acoustic isolation. “qTMS” (1303, 1304) denotes recordings with a coil within the meaning of the present invention. “Magstim” (1301, 1302) refers to a commercial 90 mm round coil.

FIG. 14 shows sound recordings belonging to the electric pulses (waveforms) from FIG. 13.

FIG. 15 shows power density spectra belonging to the electric pulses (waveforms) from FIG. 13 and the corresponding sound recordings from FIG. 14.

FIG. 16 compares the sound levels (equivalent average sound pressure level after A weighting) belonging to the electric pulses from FIG. 13.

FIG. 17 shows a circuit topology which may produce ultrabrief TMS pulses within the meaning of the invention. The circuit represents a biphasic topology, in which the conventional thyristor as switch (1702) was replaced by an IGBT. The latter permits significantly higher current dynamics, which are required for ultrabrief pulses. Possibly, future thyristor generations may also facilitate the use thereof for ultrabrief pulses. An important disadvantage of this topology is that the pulse form is fixed by the circuit to have a predetermined pulse width and hence also predetermined spectral characteristics.

FIG. 18 shows how two or more semiconductor switches may be connected in series within the meaning of one embodiment of the invention in order to increase the common dielectric strength (specified switchable peak circuit). The additional passive circuit elements form a balancing circuit which ensures that the overall voltage is divided into a plurality of stable, preferably equal parts. In this example, the resistors (Rₐ and R_{b}) divide the voltage predominantly for static voltages, for example in the state of open switches; the capacitors (Cₐ and C_{b}) stabilize the voltage divider during transient processes, for example during switching or during a sinusoidal pulse. It is likewise possible to use other known methods for dividing the voltage of series-connected switches, such as e.g. antiparallel Zener diodes and transient voltage suppressor elements.

FIG. 19 shows cTMS technology comprising a half bridge made of two electronic switches (1903, 1904). This technology permits controlling of the pulse width and may correspondingly change the frequency spectrum of a pulse in the coil L (1907).

FIG. 20 shows cTMS technology comprising two half bridges, each made of two electronic switches (2003, 2004) and (2005, 2006), for increased flexibility.

FIG. 21 shows a modular stimulator for producing high-voltage pulses by using smaller voltage steps. The figure shows the structure of the overall circuit with N modules, a coil L and a controller, as well as power supply lines. The individual modules may be implemented as small H-bridge circuits (see FIG. 22). The entire pulse voltage is subdivided into smaller units, each being approximately 1/N-th of the entire pulse voltage. The module structure keeps the circuit in equilibrium in such a way that none of the circuit components in the modules—both semiconductors and passive elements such as capacitors—are exposed to more than 1/N-th of the pulse voltage. This approach facilitates the use of cost-effective circuit elements with a low voltage rating. Moreover, the system may quickly switch between the voltage levels and synthesize pulses very flexibly and freely.

FIG. 22 shows a module circuit for the N modules from FIG. 21.

FIG. 23 shows a step-shaped pulse which may be produced by the high flexibility on account of the dynamic switching between the circuit levels of the modules from the circuit in FIGS. 21 and 22, and which may be modified from pulse to pulse.

FIG. 24 shows a random walk pulse, which illustrates the high flexibility of the circuit in FIGS. 21 and 22.

### DESCRIPTION OF THE INVENTION

The goal of the invention is to reduce the noise produced by the TMS machine and, in the process, maintain the effective strength of neural stimulation by the TMS pulse. The invention of quiet TMS consists of two parts, which may be used both in combination and individually, separately from one another.

(1) The first part consists of shifting a considerable part of the spectrum of the TMS pulse sound to higher frequencies such that the spectral component which falls into the range of highest sensitivity of the human ear between 500 Hz and 8 kHz is minimal; a shift of a considerable portion of the spectrum to frequencies above the human hearing limit of approximately 18 kHz-20 kHz is particularly preferred. This approach is based on three reasons. Firstly, the human perception of noises above the hearing limit is negligible. Secondly, mechanical oscillations are much easier to suppress from a technical point of view than those in the conventional TMS spectrum. This is based on a stronger effect of inertia, the increasing ratio of thickness of the damping means to wavelength, and the typical frequency dependence of the properties of materials for implementing the invention (see point (2) in the following sections) [Möser M., Kropp W. (2010). Körperschall. Springer, Berlin/New York.]. On the other hand, the occupational safety limits for ultrasound are higher than in the hearing range [Duck F. A. (2007). Medical and non-medical protection standards for ultrasound and infrasound. Progress in Biophysics and Molecular Biology, 93 (1-3):176-191.]. Thirdly, the required TMS pulse power is reduced for such ultrabrief pulses [Barker A. T., Garnham C. W., and Freeston I. L. (1991). Magnetic nerve stimulation: the effect of waveform on efficiency, determination of neural membrane time constants and the measurement of stimulator output. Electroencephalography and clinical neurophysiology. Supplement 43:227-237; Goetz S. M., Truong C. N., Gerhofer M. G., Peterchev A. V., Herzog H. G., Weyh T. (2013). Analysis and Optimization of Pulse Dynamics for Magnetic Stimulation. PLOS One, 8 (3): e55771.]. The use of these pulses exploits the fact that neurons may be stimulated with pulses of different form and duration if the amplitude is scaled to suitable extent. By way of example, pulses which consist of shorter electric current phases are linked to higher acoustic frequencies. Thus, if the TMS pulse phases are scaled to be suitably brief and the current amplitude is selected in a suitable manner, the dominant spectral portions of the pulse lie above the human spectral hearing range (hearing spectrum), while the pulse continues to be able to trigger neurostimulation, for example in the form of action potentials. Here, a pulse phase is part of the electric pulse; conventionally, a phase or pulse phase refers to part of the pulse during which the current's polarity does not change and which is delimited by the start of a pulse and/or the end of a pulse and/or a change in polarity of the current.

(2) The second part consists of designing the components (coil, coil cable and pulse source) in such a way that, despite the very high electromagnetic energy of a pulse, (a) only a small part of the electromagnetic energy is converted into mechanical/acoustic energy, (b) the part of mechanical/acoustic energy which is emitted to the surroundings is minimized and (c) the part of the mechanical/acoustic energy which is not emitted is quickly converted into heat within the machine. These considerations may be applied to all elements of the machine, but are most important for the stimulation coil which is the dominant source of noise on account of the high magnetic fields and electromagnetic forces and which is situated closest to the operator, subject and patient. In order to achieve targets (a-c), this invention proposes a plurality of measures, including a targeted impedance offset (also referred to as impedance mismatching), frequency-selective decoupling with phase-shifting materials and friction-afflicted elements for outputting mechanical power. According to the prior art, these measures have previously not been used in a targeted manner for improved TMS sound suppression.

Part 1: Ultrasonic Pulse Spectrum

The first part of the invention shifts a significant part of the spectrum of the acoustic emissions out of the hearing range, in particular into the ultrasonic range (>18-20 kHz). A key determinant for the acoustic emission is the waveform of the current pulse which brings about both the stimulation effect and, on account of the conversion of electromagnetic forces into acoustic oscillations, the sound emission. The first part is assisted by the second part, described further below, such that all elements must be embodied in such a way that they only convert a small portion of the energy content of the high-frequency oscillations back into the hearing range, for example by mechanical effects (for example energy exchange between modes or a nonlinear effect) and that they therefore keep the frequencies high, even in the mechanical domain.

The following reasons outline why this approach is not obvious to a person skilled in the art:

(1) The very brief electric pulse waveform does not directly set the time profile and the spectrum of the acoustic emission. While conventional pulses, to the greatest possible extent, use sinusoidal current profiles, which have pronounced spectral components with sidebands around the sinusoidal frequency (see FIG. 2), the associated acoustic sound recordings exhibit a broad, almost flat distribution of the emission along the entire audible range (see FIG. 15). The exact relationship between these two phenomena is not well understood. The large difference is due in part to nonlinear mechanical effects, which depend on the physical properties of the used materials. Further, the spectrum of conventional TMS pulse waveforms (usually so-called biphasic pulses) is not monomodal but very broad on account of the short extent and sharp attack/decay thereof.

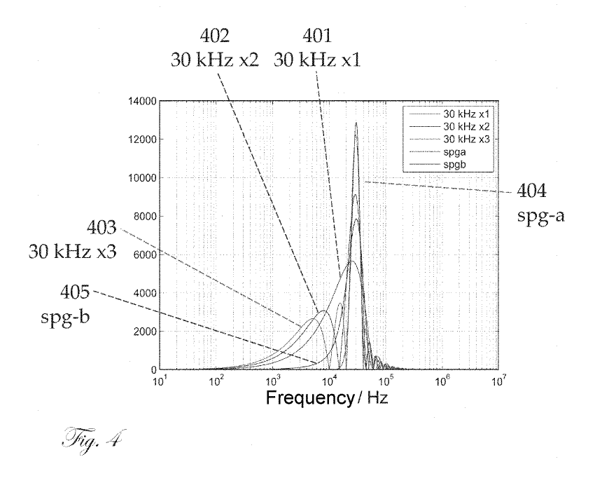

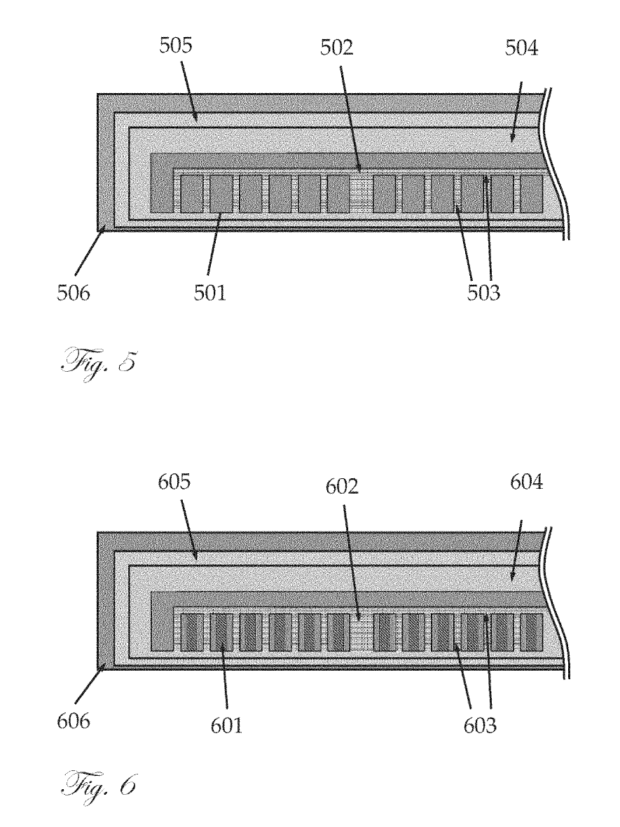

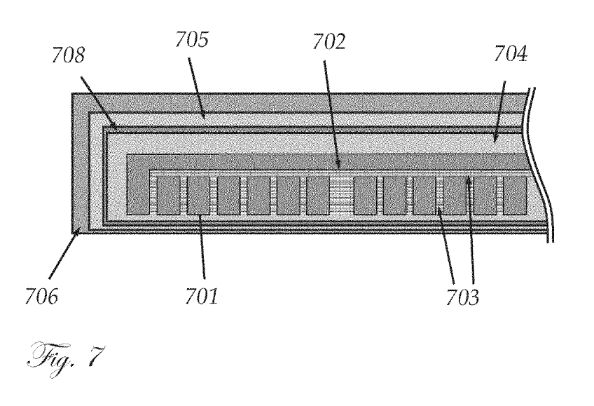

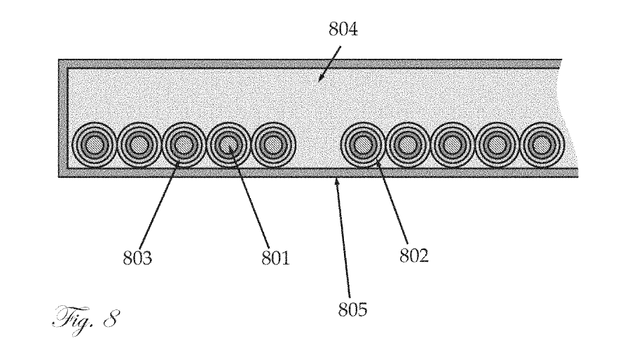

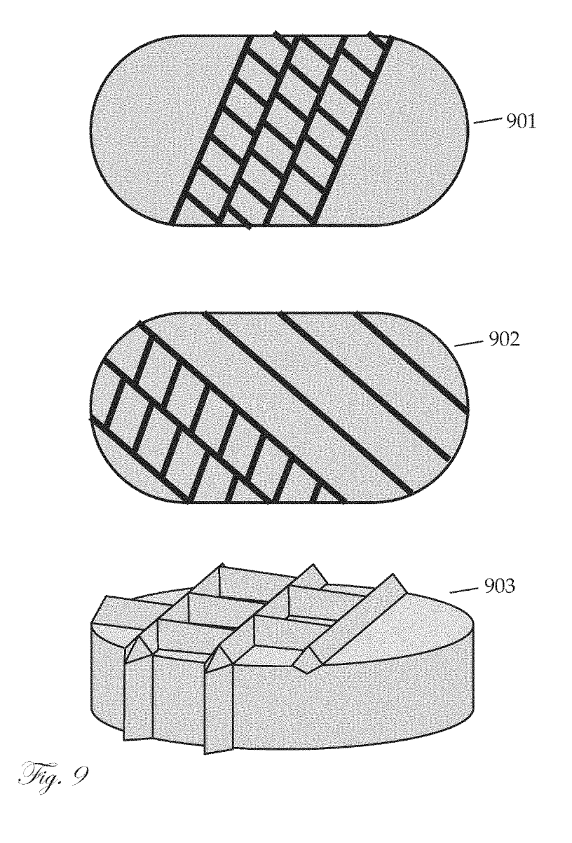

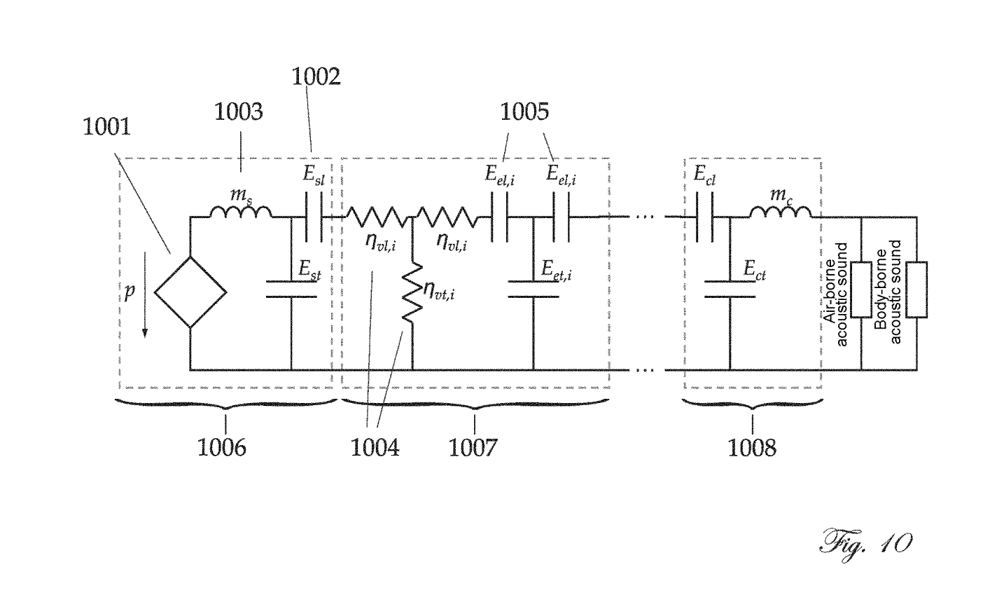

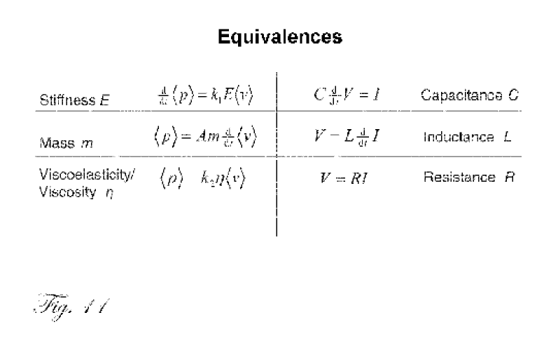

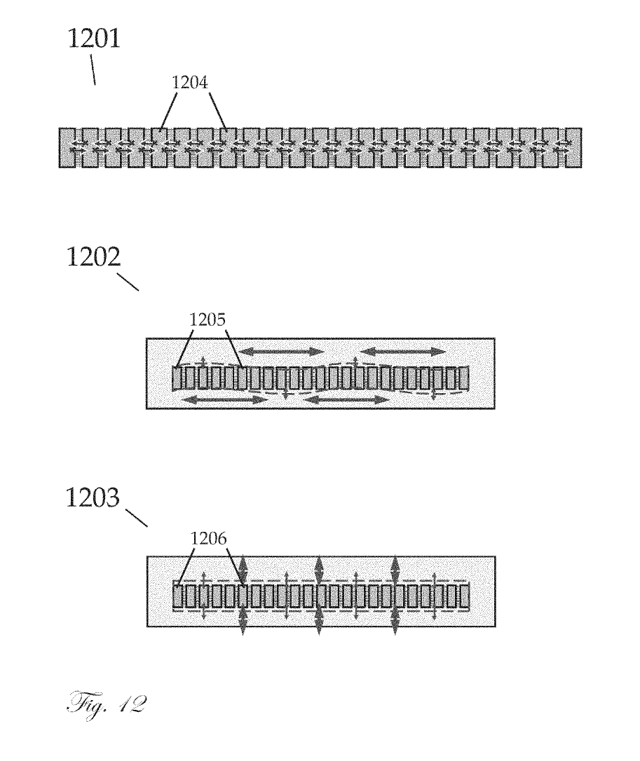

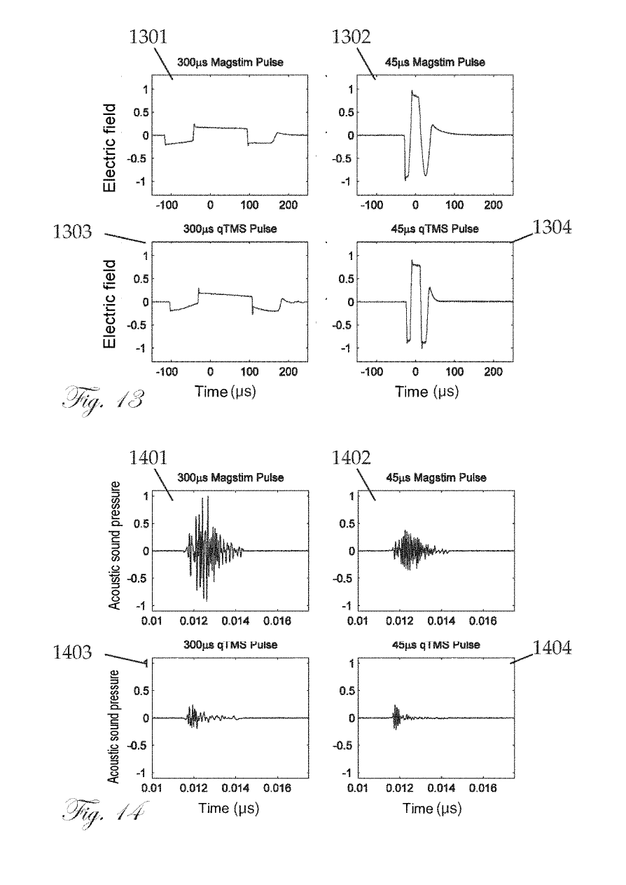

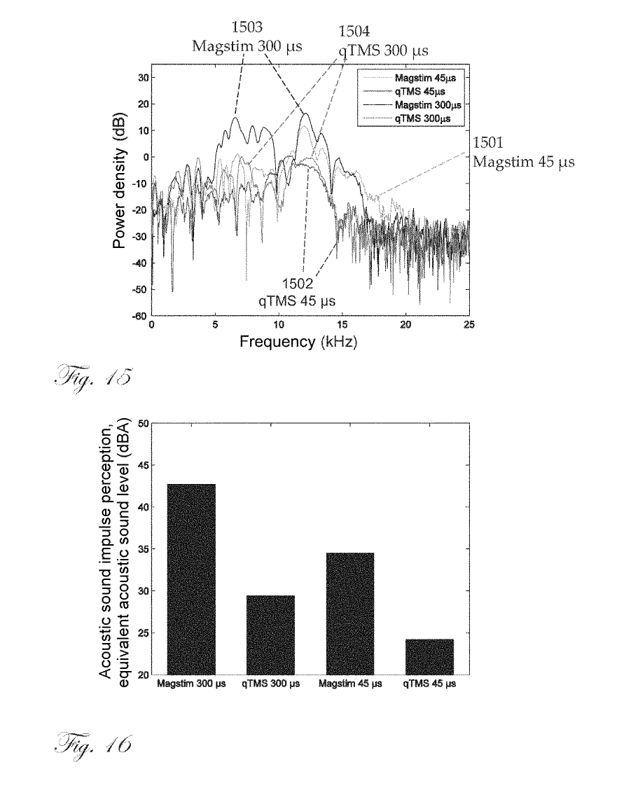

(2) The implementation of pulses for magnetic stimulation at frequencies exceeding the human hearing range was previously not possible from a technical point of view. The strong currents and high voltages required by TMS are conventionally switched using thyristors. However, thyristors are limited in their capabilities in respect of carrying out fast switching of currents. It is for this reason that existing TMS machines produce pulses which virtually exclusively lie in the range from 1 kHz to 3 kHz. This range corresponds to the highest sensitivity of human hearing and is therefore the worst in respect of noise generation. New machine technology, for example insulated gate bipolar transistors (IGBT) and metal oxide semiconductor field effect transistors (MOSFET), have only recently allowed the production of shorter pulses and more far-reaching control of the waveform. Prior to the introduction thereof, it was technically impossible or completely impractical to produce more complicated waveforms than sinusoidal waves (for example, the waveforms suggested in FIGS. 3 and 4), limiting the possibility of influencing the acoustic emissions with the aid of the TMS pulse waveform. TMS machines and associated technologies which permit fundamental control of the pulse form and pulse width were not available until they were developed by the inventors [Peterchev A. V., Jalinous R., and Lisanby S. H. (2008). A transcranial magnetic stimulator inducing near-rectangular pulses with controllable pulse width (cTMS). IEEE Transactions on Biomedical Engineering, 55(1):257-266; Peterchev A. V., Murphy D. L., and Lisanby S. H. (2011). Repetitive transcranial magnetic stimulator with controllable pulse parameters. Journal of Neural Engineering, 8:036016; Goetz S. M., Pfaeffl M., Huber J., Singer M., Marquardt R., and Weyh T. (2012). Circuit topology and control principle for a first magnetic stimulator with fully controllable waveform. Proceedings of the IEEE Engineering in Medicine and Biology Society (EMBC), 4700-4703, doi:10.1109/EMBC.2012.6347016.1.

(3) This approach is nontrivial since waveforms which largely lie of high-frequency components above the hearing range, penetrate into a range which was generally considered to be not well-suited for inductive neural stimulation, and consequently for TMS [Litvak E., Foster K. R., and Repacholi M. H. (2002). Health and safety implications of exposure to electromagnetic fields in the frequency range 300 Hz to MHz. Bioelectromagnetics, 23(1):68-82.]. However, this is contrasted by the most recent results by the inventors [Goetz S. M., Truong C. N., Gerhofer M. G., Peterchev A. V., Herzog H. G., Weyh T. (2013). Analysis and Optimization of Pulse Dynamics for Magnetic Stimulation. PLOS One, 8 (3): e55771.], which show that this treatment was, in part, a misinterpretation. Further, sound emission and control of sound sources just above the hearing range was not well-established in conventional audio engineering and technical acoustics either. The mechanical properties of most materials deviate from their behavior in the acoustic range. Both would have made an increase in the frequency difficult.

(4) On account of the shortness and the sharp onset of known waveforms in magnetic stimulation and, in particular, of the small subgroup of the latter which are able to be produced using established technology, these waveforms have a very high spectral bandwidth. It is for this reason that a shortening of the pulse was generally considered neither technically justified nor effective for reducing the acoustic sound emission. Our experimental and theoretical research supports the suggestion that the acoustic sound, to the greatest extent, is driven by the current profile and that the acoustic sound drops off with increasing frequency in comparison with the acoustic emissions, always at the stimulation threshold of a nerve.

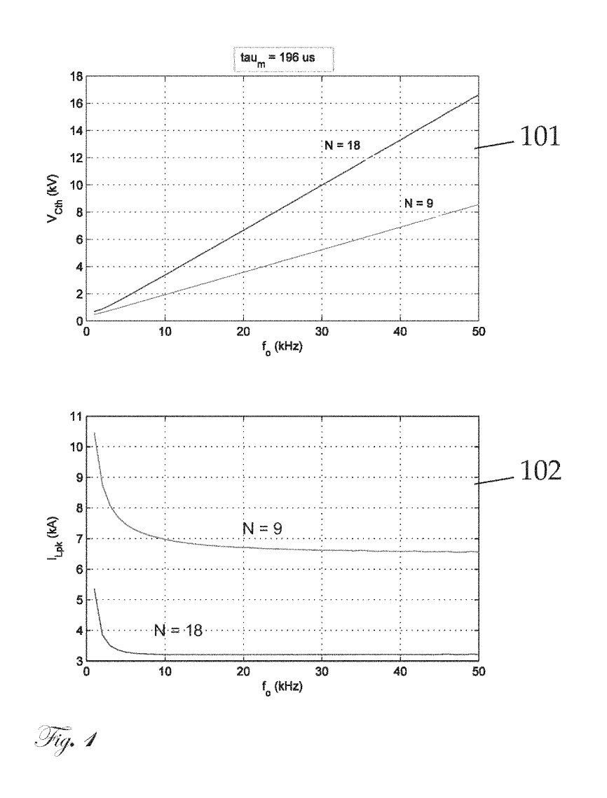

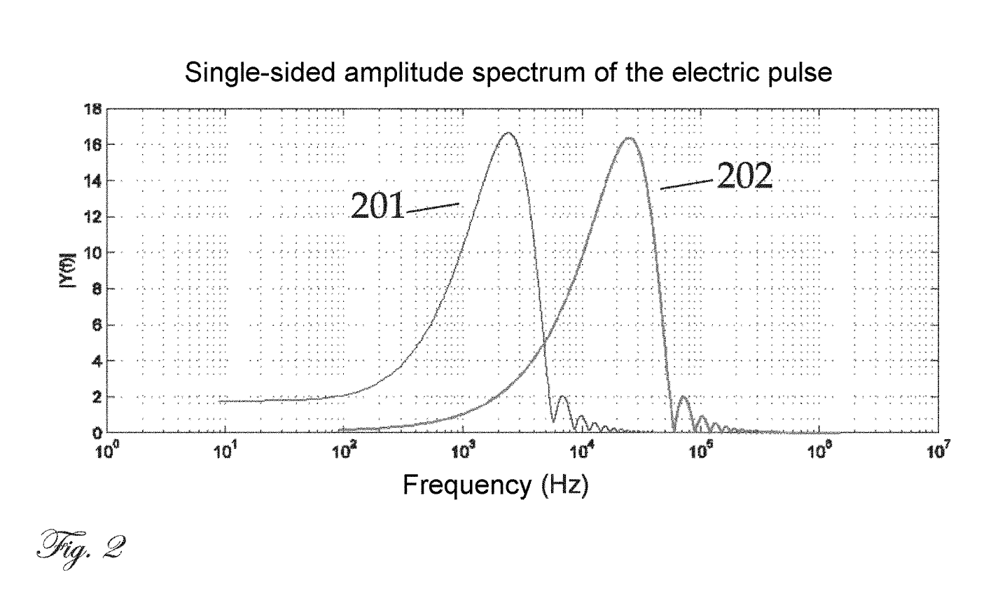

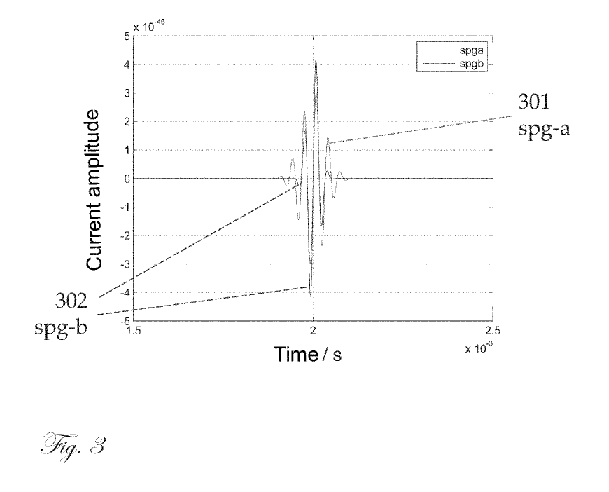

The goal of reducing the audible noise of TMS may be reached by virtue of shortening the magnetic pulse, which in turn is directly produced by the current pulse, such that the fundamental frequency and the dominant frequency lie above 18-20 kHz (see FIG. 2). A consequence of the well-known strength-pulse length relationship (the so-called strength-duration curve) of the neuronal reaction response is that the pulse amplitude must be increased in order to achieve neural stimulation of equal strength. This in turn requires the peak voltage and/or the current in the TMS coil to be increased, as shown in FIG. 1.

The magnitude spectrum in FIG. 2 compares the coil current of a conventional biphasic pulse (201, black) with the coil current of a matched pulse with a fundamental frequency of 30 kHz (202). Both stimuli were matched in a computer-assisted manner so as to have approximately the same neural stimulation strength. Although the spectral peak power is similar for both pulses, the spectral component in the hearing range is significantly reduced for the 30 kHz pulse when compared with the conventional pulse, the peak in the spectrum of which lies in the range of maximum auditory sensitivity between 0.5 kHz and 2 kHz. FIG. 16 shows that the loudness of the emitted acoustic sound is significantly reduced for shorter pulses by virtue of comparing a pulse with a duration of 45 μs to a typical 300 μs pulse, which are both amplitude-matched so as to have the same effective neural stimulation strength.

A special embodiment of the invention comprises a plurality of refinements. Instead of a sinusoidal biphasic pulse with an increased fundamental frequency—which in principle corresponds to a sinusoidal oscillation which is stopped after one period—, it is possible to increase the number of wave trains. Such multiphasic or polyphasic pulses reduce on the one hand, the neural trigger threshold [Emrich D., Fischer A., Altenhöfer C., Weyh T., Helling F., Brielmeier M., and Matiasek K. (2012). Muscle force development after low-frequency magnetic burst stimulation in dogs. Muscle & nerve, 46(6): 951-959; Wada S., Kubota H., Maita S., Yamamoto I., Yamaguchi M., Andoh T., Kawakami T., Okumura F., and Takenaka T. (1996). Effects of stimulus waveform on magnetic nerve stimulation. Japanese Journal of Applied Physics, 35:1983-1988.] and, on the other hand, the width of the spectrum compared to the biphasic pulse (see FIG. 4).

A further preferred embodiment of the invention uses an amplitude-modulated waveform which, for example, has a soft fade-in and fade-out using a Gaussian or hyperbolic-secant-shaped envelope, as are known, for example, from bandwidth-limited ultrashort laser pulses in optics (see FIGS. 3 and 4). In addition to a narrower spectrum, these pulses generally cause smaller nonlinear effects as they have a less abrupt start and, likewise, a lower peak amplitude of the magnetic field, and hence of the forces, while having the same stimulation effect compared to conventional sinusoidal, biphasic waveforms. Such nonlinear mechanical effects in the individual parts of the stimulation system, in particular in the coil, are the main mechanism which transfers the inaudible portions of a TMS waveform spectrum into the hearing range. Conventional TMS pulse source technology is unable to produce such bandwidth-limited pulses on account of restrictions linked with the circuit topology and the implementation.

The production of short pulses (i.e. with fundamental frequencies in the range of several tens of kilohertz) near the neural trigger threshold requires higher peak voltages and/or currents. FIG. 1 plots the required voltage level for sinusoidal, biphasic pulses against the fundamental frequency for two coils (9 and 18 turns). A nonlinear neuron model according to the prior art estimates a required peak voltage of approximately 10 kV for obtaining an equivalent amplitude range to a typical state-of-the-art commercial stimulator, such as e.g. the Magstim Rapid machine.

As mentioned above, the limiting factor of the most common electronic circuit, the biphasic topology (FIG. 17) for shorter pulses with a higher voltage at the same time, is the conventionally employed thyristor switch. By contrast, currently available IGBTs permit approximately 300 times higher rates of current rise. This rise in rate facilitates the ultrabrief pulse which is approximately 10 times shorter than conventional TMS pulses. Accordingly, an oscillator may produce the proposed ultrabrief pulses by reducing the product of coil inductivity L and source capacitance C.

In a special embodiment, the ultrabrief current pulses of the invention are produced using an oscillator circuit containing a pulse capacitor, an electric switch and a stimulation coil, with the switch containing at least one IGBT and the product of coil inductivity L and capacitance of the pulse capacitor C being less than 150 microhenry times microfarads.

Some TMS technologies of the inventors (see FIGS. 19, 20, 21, 22) facilitate the production of ultrabrief pulses with a sufficient voltage and several more efficient waveforms for the first time. By way of example, the pulse from FIG. 12 may be produced using the so-called cTMS technology from FIG. 13 [Peterchev A. V. (2010). US 2012/0108883, EP2432547]. The cTMS topology in FIG. 19 consists of a half bridge with capacitors Cₐ and C_{b}, which are tapped at the center. This topology is able to actively switch from one pulse phase to the next by virtue of the coil L between the capacitors Cₐ and C_{b} being commutated with the aid of switches Q₁ and Q₂. Accordingly, the pulse width and the dominant frequency and/or the fundamental frequency of the pulse may be modified by the control software, which controls the switching instances of switches Q₁ and Q₂. The capacitors Cₐ and C_{b} should have similar voltage limits in order to equally shorten the frequency of all waveform phases. The cTMS concept is extended in FIG. 20. The two half bridge circuits (Q₁-Q₂ and Q₃-Q₄) facilitate a piecewise production of the waveform with the voltage levels of the capacitors Cₐ and C_{b}, with the difference voltage thereof, and with the zero voltage level arising when the two coil connectors are short-circuited by the switches Q₂ and Q₃. The important advantage of these two circuits is that they (a) are able to produce rectangular voltage pulses, which are more efficient than sinusoidal TMS pulses and significantly reduce the peak voltage required for the stimulation [Goetz S. M., Truong C. N., Gerhofer M. G., Peterchev A. V., Herzog H. G., Weyh T. (2013). Analysis and Optimization of Pulse Dynamics for Magnetic Stimulation. PLOS One, 8 (3): e55771; Peterchev A. V., Jalinous R., and Lisanby S. H. (2008). A transcranial magnetic stimulator inducing near-rectangular pulses with controllable pulse width (cTMS). IEEE Transactions on Biomedical Engineering, 55(1):257-266; Peterchev A. V., Murphy D. L., and Lisanby S. H. (2011). Repetitive transcranial magnetic stimulator with controllable pulse parameters. Journal of Neural Engineering, 8:036016.], and (b) allow a change in the pulse width which, furthermore, sets the spectrum of the waveform (the pulse width may be controlled individually for each pulse in a pulse sequence).

An output transformer may be used for all aforementioned topologies in order to reduce the required pulse source voltage (up to ˜10 kV) to the range in which more cost-effective semiconductors are available (up to ˜3.3 kV). Here, the coil inductance may remain in the typical range of approximately 8 μH to 25 μH in order to reduce the losses on account of the otherwise (in the case of a lower inductance) higher current in the cable and an otherwise lower ratio of coil inductance to parasitic inductances in series with the coil.

An alternative approach for handling the high voltages and currents required for ultrabrief TMS pulses consists of implementing the pulse source with a modular circuit topology as depicted in FIG. 21. As shown in FIG. 21, the entire pulse voltage equals the summed output voltage of the many individual modules. By way of example, the individual modules may be implemented as an H bridge (see FIG. 22). This technology divides the entire high pulse voltage into smaller units [Goetz S. M., Pfaeffl M., Huber J., Singer M., Marquardt R., and Weyh T. (2012). Circuit topology and control principle for a first magnetic stimulator with fully controllable waveform. Proceedings of the IEEE Engineering in Medicine and Biology Society (EMBC), 4700-4703, doi:10.1109/EMBC.2012.6347016.]. Using these smaller units, the system may produce a waveform with the aid of smaller voltage steps, as imaged in the recordings in FIGS. 23 and 24. For a system with N modules (FIG. 21), the entire pulse voltage for each module is divided by N and dynamically balanced such that the system may use cost-effective low-voltage components for the switches and capacitors.

This topology may be considered to be a high-power digital-to-analog converter and may therefore produce practically any waveform. It is for this reason that this technology may produce acoustically advantageous pulses such as a bandwidth-limited polyphasic pulse with a Gaussian, hyperbolic or similarly smooth temporal envelope, as shown in FIG. 3. What is common to all employable envelopes is that they have a maximum level, from where the envelope decreases monotonically on both sides, with the absolute magnitude of the derivative not exceeding a predetermined limit. A justifiable value for this limit is the amplitude of the envelopes divided by the period length of the polyphasic pulse.

Part 2: Acoustically Improved Coil

While the ability of reducing the acoustic emission by using suitable pulse forms is encompassed by part 1 of the invention, part 2 relates to the mechanical design of the system. This includes the conversion of electromagnetic energy into the mechanical domain, the propagation (also as diffusion or transfer), conversion to heat and emission as air-borne acoustic sound and body-borne acoustic sound, i.e. the clicking which is usually linked to TMS pulses. As already mentioned, the mechanical system needs to satisfy two conditions. Firstly, the conversion and acoustic emission should be minimal. Secondly—if this should be combined with part 1 of the invention—, the acoustic sound spectrum of the clicking should be kept above the hearing range. This includes a minimization of nonlinear mechanical effects which produce new frequency components by waveform distortion. Further, the frequency-dependent acoustic impedance should be designed in such a way that all acoustic oscillations in the hearing range are kept within the TMS machine (including the coil) such that they can be converted into heat there.

Below, the complete path of the acoustic waves and oscillations is subdivided into a plurality of sections, which are treated with different means. The acoustic path ranges from the source (all parts which directly conduct the pulse current) to the surface of the machine, where the acoustic sound is mechanically coupled to the subject or patient as body-borne acoustic sound and/or emitted into the surroundings as air-borne acoustic sound. On account of the strict size and weight restrictions, the coil and the coil cable are the most important machine parts in view of the emissions. By contrast, the pulse source may easily be damped using known, conventional acoustic sound protection measures. Even though the text focuses on the coil as an example, the invention may be applied to all elements of a stimulation system.

The coil structure of the invention systematically decomposes the acoustic path into three parts. (1) The acoustic source in a coil is the electric conductor which vibrates on account of the magnetic forces which are produced by the high pulse currents. The central process in this case is a conversion of some of the electrical pulse energy into mechanical energy, wherein it is this conversion that should be minimized. (2) Further, the transfer of the acoustic energy, now reduced by (1), to the surface, where it is emitted to the surroundings as air-borne acoustic sound and as body-borne acoustic sound, should be suppressed. (3) Instead, a significant portion of the acoustic sound may be suppressed by virtue of being converted into heat by means, provided to this end, in the coil. On the basis of this decomposition, the invention reduces the entire acoustic sound output by adjusting the mechanical impedance with the aid of impedance mismatching, phase-shifting elements (materials with a high elasticity and mass density) and (phase-neutral) frictional-loss-afflicted material properties (viscoelasticity).

At the coil conductor, the system may be considered to be an energy transducer which couples two domains: the electromagnetic domain and the acoustic/mechanical domain. In contrast to conventional sound engineering, in which the sound source usually cannot be modified, the conversion process may be included in the solution to the problem for TMS machines. The acoustic sound sources in magnetic stimulation systems are the conductors which conduct the high stimulation pulse current. On account of the electromagnetic forces within and between conductors, some of the electric energy is converted into acoustic energy. In order to reduce this conversion such that further damping measures only have to overcome a minimum of acoustic energy, the acoustic impedances are deliberately mismatched/offset in relation to one another (also referred to as impedance matching). The sound source (i.e. the electromechanical transducer) is a pressure-excited source (equivalent to a synchronous motor below the breakdown torque, which is therefore in the force-excited range). This implies that the mechanical pressure amplitude at the conductors is virtually constant, while the resulting deflection depends both on the pressure and the mechanical impedance. Accordingly, the incoming acoustic oscillations are presented with a high mechanical input impedance in the invention in order to minimize the conversion rate. Further, this step may be used, in particular, to suppress lower frequency modes in the audible spectrum. Hence, the nonlinear conversion of high-frequency excitation into audible components may be reduced further.

### Energy Conversion Section

A central aspect of the first section of the acoustic path lies in reducing the conversion of electric energy into acoustic oscillations. Firstly, this step minimizes the portion of energy which needs to be subsequently damped. Secondly, the portion of energy which is not converted now remains on the electrical side of the electromechanical transducer and is no longer part of the losses, as a result of which the efficiency of the TMS machine is also increased.

The conversion efficiency is substantially a mechanical impedance problem. The conversion of electric energy into mechanical vibrations occurs at the power current conductors in the coil, the pulse source and the cables therebetween, which vibrate due to the alternating magnetic forces. In conventional TMS systems, the electrical side is formed by a high-voltage power current oscillator with a low mechanical energy loss. Future TMS technologies, which do not implement an oscillator circuit, will, with a high probability, continue to have a low impedance and a low mechanical energy loss. Accordingly, the conductors behave as a pressure source from a mechanical point of view. Consequently, the electric source may be considered to be inexhaustible from a mechanical point of view and all damping measures which conventionally work toward converting vibrations into heat need to be avoided in order to obtain a low conversion rate. Like a voltage source connected to a low load resistor, the sound pressure would only be reduced slightly while there would be a strong increase in the acoustic sound speed —as the acoustic equivalent of the electric current—and hence in the acoustic energy which would be converted into the mechanical domain. Only very strong damping could exhaust the energy source such that the acoustic emission would drop again. However, as a rule, this would mean a virtually complete conversion of the electrical pulse energy of the TMS system into the mechanical domain.

It is for this reason that, in accordance with the present invention, the conversion is reduced in a targeted manner by increasing the mechanical impedances in one or both of the following ways:

(a) The mechanical stiffness of the conductor compound is increased. In particular, this measure prevents the occurrence of low-frequency and mid-frequency acoustic components. Since, for constant power, the acoustic sound speed falls approximately with the inverse of frequency and since the stiffness of most materials falls nonlinearly with frequency (i.e. the mechanical impedance increases with frequency), the influence of the stiffness is reduced for higher frequencies. Nevertheless, a higher stiffness shifts the conversion range to higher frequencies, suppresses the conversion of spectral sidebands and prevents the formation of audible low-frequency components as a result of nonlinear effects.

Measures for increasing the stiffness of the conductor include, for example, the use of bimetal structures made of copper (or other materials with a good electrical conductivity) and a stiffer metal, a braced conductor embedded in a stiffer material, stiffening elements such as beams or struts and/or the connection of different conductors or parts of conductors with rigid structural adhesives. Steel is a suitable stiffening material; it has a Young's modulus (E-modulus) approximately four times higher than that of copper [Moser M., Kropp W. (2010). Körperschall. Springer, Berlin/New York.]. Thin flat conductors as are used, for example, in commercially available coils are suboptimal without rigid stabilization. For the measure of increased stiffness, the frequency transfer function follows approximately 6 dB 1d(f)/1d(E) with the frequency f, the stiffness E and the binary logarithm 1d. Further, most real materials have a frequency-dependent stiffness, which, as a rule, increases with frequency [Möser M., Kropp W. (2010). Körperschall. Springer, Berlin/New York.].

(b) The mass of the conductor compound is increased so as to increase inertia. While the stiffness predominantly prevents the conversion of lower frequencies and shifts the frequency transfer function and possible resonances to higher frequencies, the mass limits the spectrum in the high-frequency range by suppressing fast deflections and hence the acoustic sound speed. If the electromagnetic spectrum of the TMS pulse is selected predominantly in the high-frequency range, the influence of the mass is significantly increased. The frequency characteristic of the impedance approaches exponential growth with a growth rate of 6 dB/[1d(m) 1d(f)], with a mass m and the frequency f.

A suitable material selection and/or an increase in the volume are ways for increasing the effective masses. Accordingly, a large cross section of the conductor and high-density materials are advantageous. By contrast, completely replacing the copper with more dense conductors may usually not be economical because the conductivity of these stiffer materials is significantly less than that of copper (by factors of two to approximately ten). It is for this reason that these stiffer materials are most advantageous at points of the conductor where the current density is low, for example on account of high-frequency effects and other current displacement phenomena.

### Acoustic Sound Conduction Section

A second aspect when designing the acoustic path lies in the reduction of the propagation of acoustic oscillations from the conductors to the surface, from where said oscillations are emitted to the air as acoustic sound and/or to the subject as body-borne acoustic sound.

The dominant mechanical modes for the frequency range, the spatial extent and the characteristic wave speed of the materials of TMS coils are predominantly represented by bending oscillations. By contrast, transverse shear waves and longitudinal pressure waves occur predominantly at the lower end and upper end of the frequency range relevant here, where they may be reduced efficiently using known methods.

Accordingly, it is necessary to consider the wavelength range and the propagation mechanisms of the acoustic emission. These depend on the geometric extents, the material properties (in particular the wave speed for the specific vibration type) and the excitation frequency, which is set by the electromagnetic waveform. The dominant components are usually specified by bending oscillations for the typical conditions in TMS. Additionally, high-frequency components may emit surface waves. The coil may act as a point source only in the lower frequency range of the oscillations and, in particular, for relatively small coils. Instead of bending, the entire body carries out almost uniform oscillations in the form of translation or contraction/expansion in this case and will be similar to a loudspeaker. For the materials and the compact structure of coils, such a point source behavior would appear to occur merely at the lower end of the human hearing spectrum below 1 kHz.

As a result, the material layers represent not only a specific impedance on the path from the inside to the outside, i.e. from the source to the surroundings, but also an impedance perpendicular to this direction, along a layer. An acoustic energy flow along this direction is a consequence of the inhomogeneous instantaneous sound pressure conditions on account of different modes. A targeted use of material properties such as stiffness, mass, viscosity and elasticity is used in this invention to control different acoustic sound components. In a simplified picture, stiffness and viscosity are e.g. most effective against dominant bending vibrations and bending waves, while inertia suppresses the lower frequency point-source-like components.

The phase-shifting, capacitive nature of an elastic path to the surface decouples the coil winding block (core) and suppresses the transfer of the acoustic energy to the surface of the coil by way of a low-pass property. Dissipating the captured mechanical energy as heat is achieved in a stiff viscoelastic layer which may cover the acoustic source (the conductor core) as an energy-dissipating shunting resistive path.

### Layer Thicknesses, Electrical Insulation and Safety

The TMS coil conductor may be encased in high-voltage electrical insulation. The acoustic materials may also be used for electrical insulation. A relatively thick layer made of insulation material between the coil and the head of a subject may improve the acoustic properties of the coil. However, an increase in the insulation thickness requires higher pulse amplitudes for stimulation purposes and accordingly generates more acoustic sound in the turns. It is for this reason that the viscoelastic layers and the elastic layers should only have thicknesses of the order of one millimeter per layer on the side facing the subject. By contrast, on the edges of the coil and on all other sides, conventional coil types such as figure-of-eight coils (also referred to as butterfly coils) and round coils may provide thicker (acoustic and electric) insulation.

Like in the case of conventional TMS coils, the coil insulation has two aspects. The insulation between the individual coil turns is not relevant from a safety point of view and may therefore be embodied as simple standard insulation pursuant to IEC 60601. Usually, insulation materials are further selected in such a way that they are arc resistant (e.g. level 4 pursuant to VDE 0303) in order to avoid side effects of possible breakdowns. Potting products with dielectric strengths of more than 20 kV/mm are available for all proposed mechanical materials, said potting products including elastic silicone at 25 kV, highly stiff epoxy composites at 33 kV/mm, polyurethane (PU) at 35 kV/mm, polyethylene terephthalate (PET) at 90 kV/mm, acrylonitrile butadiene styrene (ABS) at 70 kV/mm. Therefore, adjacent turns which are only exposed to a fraction of the overall voltage (as a rule, less than 1 kV) may be sufficiently insulated by the core potting. Insulation spacings of up to 1 mm should be considered at locations where turns with a relatively high voltage difference encounter one another.

By contrast, the insulation between the conductor and the surface is considered to be relevant from a safety point of view and should therefore be reinforced insulation pursuant to IEC 60601. Insulation strengths of more than 25 kV (AC) with an overall thickness of more than 2.5 mm are proposed with the insulation properties of the materials which were proposed for the mechanical design.

Even though ultrasonic emissions are not audible, they may nevertheless have negative impacts on humans. However, such high-frequency oscillations are relatively easy to suppress as the effect of all three methods described above increases with increasing frequency. The increasing effect is a consequence of the inertia, the increasing ratio of layer thickness to wavelength and/or typical ones of the typical frequency dependence of the material properties, and also shows in prototypes. For this reason, the ultrasonic emissions may be kept below occupational limits (110 dB +9 dB; [Duck F. A. (2007). Medical and non-medical protection standards for ultrasound and infrasound. Progress in Biophysics and Molecular Biology, 93 (1-3):176-191; ACGIH (2001). Documentation of the Threshold Limit Values for Physical Agents. Cincinnati (Ohio).]) and recommended public exposure limits (100 dB; [Duck F. A. (2007). Medical and non-medical protection standards for ultrasound and infrasound. Progress in Biophysics and Molecular Biology, 93 (1-3):176-191.]), both of which are lower than the limits for medical products pursuant to IEC 60601, with little outlay.

### Detailed Description of the Layers

As described above, two layers—a viscoelastic layer and an elastic layer—reduce and conduct the acoustic sound emission of the preferably stiff and heavy conductor core. Preferably, the viscoelastic layer covers the core, while the elastic layer surrounds the viscoelastic layer.

Here, as a rule, a layer or material layer is a volume, filled with at least one material in any known state of matter (e.g. a low-pressure gas or low-pressure gas mixture as well), with the volume having at least one well-defined surface which is in mechanical contact with at least one other material and the interface formed by the contact having a finite area, preferably greater than one square centimeter, particularly preferably greater than five square centimeters. Here, the interface between two materials should suppress mixing of the materials. By way of example, two liquids or gases that are soluble in one another cannot form an interface within the meaning of the invention. By contrast two solid-state materials (including materials which are often assigned to the class of soft matter, for example polymers, gels, material foams, etc.), for example, may form layers with a well-defined interface, even if slow material degradation, material diffusion or the like from one material layer into the respective other one leads to a gradual material transition instead of a step-like material transition, provided the process of mixing at the interface during operation is slow compared with typical operating durations, there preferably being less than 1% mass diffusion into the respective other material per hour. The minimum volume of a material layer is preferably 100 cubic millimeters. A layer or material layer need not necessarily be contiguous, but may also consist of a certain number of individual parts or individual spots which are arranged next to one another, for example with gaps. Furthermore, a layer or a material layer may contain a plurality of different materials which provide the desired overall property (e.g. stiffness, viscoelasticity or elasticity) together, in combination, or each material of which generally has the desired property, but to a different extent or different strength in each case.

(i) Viscoelastic Layer:

The viscoelastic layer is distinguished by a high viscosity η. Ideally, the latter is accompanied by a high stiffness on account of a high Young's modulus. The product Eη facilitates both the suppression of bending modes and a conversion into heat, leading to dampening of the acoustic sound waves/acoustic sound oscillations which enter into the layer. To this end, it is advantageous if the viscoelastic layer has a mechanically secure connection to the adjoining layer situated closer to the source. In this case, bending oscillations and bending waves of the core may be reduced by frictional losses as a result of a shearing load, which represents the most effective mode for most viscoelastic materials. Although this is not mandatory without exception, the effect of the viscoelastic layer may be significantly increased if it is terminated by a stiff and possibly (but not necessarily) high-mass layer which is configured in such a way that the viscoelastic layer is bounded on both sides. The stiff winding conductors or any other stiff layer adjoining the viscoelastic layer from the inside and the additional stiff layer on the outer side may, together, advantageously increase the energy losses significantly on account of a shearing load.

(ii) Elastic Layer:

In contrast to the winding conductor, which represents the source of the acoustic oscillations and which is distinguished by an approximately constant pressure amplitude and a low impedance, the interface of the viscoelastic layer to adjoining layers acts as a source with a high source impedance which may be exhausted by all means. That is to say, the energy content thereof is practically exhaustible. It is for this reason that decoupling by a highly elastic layer is possible. The elastic layer does not suppress the oscillations but acts like the mechanical equivalent of a (phase-shifting) capacitor in the electrical domain and produces a mechanical low-pass filter. The characteristic equations of the capacitor and of the elastic layer are similar here: d/dt<p>=K E <v>, where the pressure p represents the equivalent of the electric voltage, the speed v is the equivalent of the electric current, E is the stiffness and K is a constant of proportionality.

Should it be possible to change the mass/mass density of one or both layers, the impedance mismatching effect may be significantly increased by a high density/mass of the viscoelastic layer. By contrast, a low density/mass is generally advantageous for the elastic layer. The inner core is mechanically decoupled from the casing by way of the elastic layer.

The effectiveness of both the elastic layer and the viscoelastic layer increases with increasing frequency. This effect is increased further by the nonlinear behavior of the viscosity of many materials (known as a dilatant property, see [Möser M., Kropp W. (2010). Körperschall. Springer, Berlin/New York.]). Accordingly, a displacement of the electromagnetic waveform to higher frequencies within the meaning of the invention also simplifies acoustic damping.

To ensure that the decoupling approach works correctly, an elastic layer should be surrounded by a high-mass and/or stiff layer. This may be either the casing of the coil or a repeating sequence of viscoelastic and/or elastic layers, followed by a casing. In order to increase the mass density and the stiffness of the casing, use may be made of fiber reinforcement, plastic molds (e.g. duroplasts), acrylamide polymer composites, ceramics or composites consisting of a polymer with inorganic fillings.

To a first approximation, the coil arrangement presented above (which may likewise be applied to the pulse source and cables) may be represented by a much simplified equivalent circuit as shown in FIG. 10. The equivalent circuit consists of a pressure source p, a deliberately high source impedance represented by the mass mₛ and the high stiffness Eₛ, a damping block consisting of a highly elastic (i.e. less stiff) element Eᵢ and the viscoelastic component ηᵢ (both may be repeated), and the casing with the mass m_{c} and the stiffness E_{c}.

### Further Embodiments

The two main embodiments of the concept described above with regards to a quiet mechanical design differ in the embodiment of the individual elements, in particular in the conductor/conductors of the turns. Differences in the capability also depend on the frequency range and the dominant type of the acoustic modes.

In a first embodiment (see e.g. FIG. 5), some or all of the turns of the conductor or conductors are combined in a single stiff block and securely connected to one another mechanically. The individual turns are closely connected, for example embedded in an epoxy matrix. Since the compression forces on a conductor are directed to the neighbors, it is further possible to establish close mechanical contact between the turns and/or produce increased rigidity and stiffness by mechanically braced conductors.

The stiff winding block suppresses mechanical movement and increases the input impedance from the perspective of the electrical pulse source and the pressure source as secondary side of the electromechanical energy transducer. The whole winding block is subsequently damped and decoupled by a combination of viscoelastic and elastic layers, the sequence of which may be repeated, as described above. The casing may follow either an elastic or a viscoelastic layer. The advantage of this embodiment is that the conductor block, which acts as an acoustic source, may easily be reinforced and stiffened using various types of known measures, such as e.g. beams/struts or fiber composites (e.g. glass fibers or polyamides), and suitable conductor forms in such a way that possible acoustic modes or frequency windows are shifted to higher frequencies. The entire conductor block may be relatively compact and requires little space. However, the small distance between the individual turns requires suitable electrical insulation which, under certain circumstances, may adversely affect the stiffness.

In a second embodiment (see e.g. FIG. 8), each turn is decoupled separately. Accordingly, each turn is surrounded by at least one viscoelastic layer and one (optional) elastic layer. In contrast to the first embodiment described above, this embodiment requires more space but is less critical in respect of electrical insulation between turns and a possibly insufficiently stiff mechanical connection between individual turns. The insulation requirements may be important for ultrabrief, high-frequency pulses, which have a significant component of their electromagnetic pulse spectrum above the hearing range and which require comparatively high voltages of several kilovolts, as explained above.

Further, the two embodiments described above may form a hybrid form, which unifies the advantages of the two embodiments above, as further embodiment. In this hybrid form, each individual turn has at least one separate elastic and/or viscoelastic layer, like in the second embodiment. Additionally, two or more or all turns share the remaining layers, like in the first embodiment.

The mechanical source impedances may be increased further in a preferred embodiment. As explained in detail, the source impedance may be increased by increasing the stiffness (described by Young's modulus) and/or the mass m. Since the high electrical conductivity of copper is advantageous in the conductor, use may be made of additional materials for changing the mechanical conductor properties. While this may also be achieved by way of an alloy, possibly with spatially heterogeneous materials, this embodiment prefers bimetals and copper-clad metals. Such conductor connections are by two or more metals—of which at least copper or a material (for example silver or gold) with a certain purity which conducts similarly well—which are securely connected to one another mechanically. The tight and mechanically secure connection may be produced using known methods, for example various welding techniques or chemical methods such as electroplating.

Such copper-clad conductors are used in a number of power engineering applications for the purposes of saving copper. In order to increase the stiffness of the conductor, a particularly preferred embodiment uses copper-clad steel conductors. These conductors and the interface between the individual, usually metallic components may be selected to be any geometric form. For this particularly preferred embodiment, the copper component is advantageously selected in such a way that it reflects the unequal local current distribution in the conductor cross sections on account of skin effect and proximity effect and other current displacement phenomena in such a way that the copper, with its good conduction, is placed at the locations of high current density. Accordingly, the effective conductivity of the whole conductor is only slightly lower than that of a pure copper conductor, despite significant advantageous acoustic properties on account of the increased mechanical stiffness. Molybdenum and tungsten, which both have a higher Young's moduli and a higher mass density, are alternatives to steel. In addition to the increased overall stiffness of the conductor, the different Young's moduli of the individual components in such a conductor compound lead to different acoustic sound speeds and may therefore prevent standing wave modes. Further, the material costs of the coil, which were previously dominated mainly by copper, are reduced.

Since the frequency components of the electromagnetic pulse are relatively high such that the skin effect and the proximity effect, as high-frequency current displacement phenomena, play an important role, the conductor may, in a further particularly preferred embodiment, be subdivided further into smaller subregions or filaments, as are known from high-frequency litz wires, such that the entire conductor cross section is subdivided into smaller units, which are embodied either electrically insulated from one another or with poor conduction. The litz wire principle of this particularly preferred embodiment reduces the frequency-dependent increase in the conductor resistance and may be achieved in this application by structuring the conductivity of the conductor into subdivisions of the cross section with different conductivity. The two or more components of the compound conductor, for example copper and steel, may be structured in such a way that the material with good electrical conduction forms a plurality of independent current paths along the conductor or the conductor axis, which is mechanically securely embedded into the mechanically stiffer material which is less electrically conductive, similar to a litz wire with a plurality of filaments.

Alternatively, a further preferred embodiment uses a high-frequency litz wire. In this embodiment, the aforementioned concept of a stiff coil winding as an acoustic source recommends that the litz wire is embodied as stiff as possible. By way of example, this may be achieved by virtue of the litz wire being embedded in a stiff material such as e.g. a ceramic or a polymer. Furthermore, the individual filaments of the litz wire itself may be compound conductors, for example copper-clad steel. In the latter case, the individual filaments receive a high stiffness on account of the material properties.

Although less capable, a reduced acoustic sound emission compared to solutions known from the prior art may be achieved in a further embodiment of the invention by virtue of use being made of either a viscoelastic layer or an elastic layer only. This embodiment uses only one mechanism, either a decoupling of the conductor core from the casing or an increase in the mechanical losses, for reducing the acoustic sound emissions. However, this case also provides a reduction in the conversion of electric energy into mechanical energy by increasing the stiffness and/or the mass density and/or the mass of the core.

A further embodiment refers to a method for stimulating neurons and/or myocytes, wherein magnetic field pulses which cause stimulating electric currents in the body tissue according to the principle of electromagnetic induction are produced by current pulses, said stimulating electric currents triggering an action potential of the neurons and/or myocytes, wherein the magnetic field pulses are produced by a coil which is positioned so closely to the body tissue to be stimulated that the magnetic field produced by the coil penetrates through the body tissue, and wherein the magnetic field pulses have a temporal profile which corresponds to a temporal profile of an electric current through the coil; and wherein the temporal current profile during a strong current pulse in the coil is selected in such a way that less than a quarter of the energy of the current pulse lies in the spectral range from 500 Hz to 18 kHz.

A further embodiment of the invention produces short strong current pulses with an overall duration of less than 1 ms in at least one coil such that the at least one coil produces magnetic field pulses with a magnetic flux density of 0.1 to 10 Tesla which cause electric currents in the body tissue according to the principle of electromagnetic induction, said electric currents triggering an action potential of neurons and/or myocytes by a stimulus, wherein the at least one coil is embodied in such a way that it is positionable close to the body tissue to be stimulated so that the magnetic field produced thereby penetrates the body tissue; wherein the device contains at least one capacitor for storing energy required for the magnetic field pulses, wherein the stimulating electric currents caused by the magnetic field of the coil are at at least one tenth of and at most ten times the stimulation currents required for stimulating the cells. This embodiment is characterized in that, for the purposes of reducing the acoustic sound emitted on account of the current pulse by the coil and/or at least one electric connection cable to the at least one coil, it is embodied in such a way that at least one electric conductor of the at least one coil and/or of the at least one electric connection cable forms a stiff unit by being embedded into a mechanically stiff polymer and/or a mechanically stiff plastic and/or a mechanically stiff composite and/or a mechanically stiff ceramic and/or a mechanically stiff glass.

In a special embodiment, the reduction in the emitted acoustic sound in the preceding embodiment consists of a reduction in the psychoacoustic loudness and/or the peak acoustic sound level and/or the acoustic sound energy and/or the psychoacoustic roughness and/or the psychoacoustic sharpness.

In a special embodiment, the at least one coil and/or the at least one electric connection cable of the preceding embodiments further contains at least one viscoelastic material collection and/or at least one elastic material collection.

In a special embodiment, at least one conductor of the at least one coil and/or of the at least one electric connection cable of one of the preceding embodiments comprises at least two different metals, which may also be different alloys in each case, wherein the at least two metals have at least one interface at which the at least two metals are securely connected to one another mechanically, wherein at least one of the at least two metals has an electrical conductivity which is at least twice as high as that of at least one other metal of the at least two metals, and, at the same time, a Young's modulus which is at most half the size of said at least one other metal of the at least two metals.

In a special embodiment, the at least two metals are arranged in the cross section of the at least one conductor of the preceding embodiment in such a way that the metal with the highest electrical conductivity of the at least two metals is preferably arranged in regions with a high current strength and at most one third of the electric pulse current, which is not uniformly distributed over the cross section of the at least one conductor on account of skin effects and other current displacement phenomena, flows in that one of the at least two metals which has the lowest electrical conductivity.

In a special embodiment, the Young's modulus of the at least one elastic material collection in one of the preceding embodiments is less than one eighth of the Young's modulus of the mechanically stiff polymer and/or of the mechanically stiff plastic and/or of the mechanically stiff composite and/or of the mechanically stiff ceramic and/or of the mechanically stiff glass.

In a special embodiment, the product's viscosity and Young's modulus of the at least one viscoelastic material collection from one of the preceding embodiments exceeds 10 billion pascal-squared seconds.

In a special embodiment, the loss factor of the viscoelastic material pursuant to ISO 6721, measured using a 2 mm material coating of the viscoelastic material on a 1 mm thick steel sheet, exceeds 0.75.

In a special embodiment, at least one viscoelastic material collection covers at least one third of the surface of the stiff unit of one of the preceding embodiments, wherein the stiff unit is formed by the at least one conductor embedded in a mechanically stiff polymer and/or in a mechanically stiff plastic and/or in a mechanically stiff composite and/or in a mechanically stiff ceramic and/or in a mechanically stiff glass. Further, the viscoelastic material is connected to this surface in a mechanically adhesive manner, wherein the viscoelastic material collection may be surrounded by further other material collections.

In a special embodiment, at least one elastic material collection covers at least one third of the surface of the stiff unit of one of the preceding embodiments and/or at least one viscoelastic material collection partly covering the aforementioned stiff unit from one of the preceding embodiments, wherein the stiff unit is formed by the at least one conductor embedded in a mechanically stiff polymer and/or in a mechanically stiff plastic and/or in a mechanically stiff composite and/or in a mechanically stiff ceramic and/or in a mechanically stiff glass.

In a special embodiment, an elastic material collection, which may form a layer, covers at least part of that surface of the coil which has mechanical contact to the body tissue.

In a special embodiment, the at least one elastic material collection from one of the preceding embodiments consists of a material which is assigned to the class of soft matter;

or consists of gas;

or consists of a vacuum;

or consists of a mixture of a solid and/or a material from the class of soft matter and a gas;

or consists of a mixture of a solid and/or a material from the class of soft matter and a vacuum;

or consists of a liquid;

or consists of a polymer foam;

or consists of a mixture of a solid and/or a material from the class of soft matter and a liquid.

Further, the at least one elastic material collection may comprise a spring mechanism, produced from a solid, in a gas and/or a vacuum.

In a special embodiment, the material of the at least one elastic material collection from the preceding embodiment is an elastomer and/or a polymer melt and/or a gel and/or a colloidal suspension.

In a special embodiment, less than one quarter of the energy of the electric current pulse of one of the preceding embodiments lies in the frequency range from 500 Hz to 8000 Hz.

In a special embodiment, the fundamental frequency and/or the dominant frequency of the electric current pulse of one of the preceding embodiments lies higher than the human hearing limit of 18 kHz.

In a special embodiment, less than one third of the energy of the electric current pulse of one of the preceding embodiments lies in the frequency range below 18 kHz.

In a special embodiment, the electric current pulse of one of the preceding embodiments contains exactly one zero crossing, at which the current switches from one polarity to the other, and wherein the overall duration of the current pulse does not exceed 75 microseconds.

In a special embodiment, the electric current pulse of one of the preceding embodiments comprises a sinusoidal oscillation, which may have a finite duration or an infinite duration and the amplitude envelope of which increases from less than one fifth of the maximum to a maximum in less than 500 microseconds and subsequently drops back down to less than one fifth of the maximum in less than 500 microseconds, wherein the frequency of the sinusoidal oscillation may change continuously during the current pulse.

In a special embodiment, the electric current pulse of one of the preceding embodiments is produced by an electric pulse source which contains one at least three capacitors and which produces a current pulse by dynamic electric combination (for example electrically conductive in series and/or in parallel) of the at least three capacitors, wherein the electric pulse source is able to produce current pulses with different amplitude and form, wherein the amplitude and the form may be changed independently of one another between the production of two successive current pulses.

In a special embodiment, the electric current pulse of one of the preceding embodiments is produced by an electric pulse source which contains at least one capacitor (1701, 1901, 1902, 2001, 2002) and at least one electronic switch (1702, 1903, 1904) (e.g. an IGBT) which can be switched off.

In a special embodiment, the electric current pulse of one of the preceding embodiments is produced by an electric pulse source which at least one capacitor (1901, 1902, 2001, 2002) and at least two electronic switches (1903, 1904, 2003, 2004) (e.g. IGBTs) which are connected electrically in series and which can be switched off, wherein the electric connection between the at least two electronic switches which can be switched off is connected by way of at least one third electric connection to at least one connector of the coil (1907, 2007), either directly or indirectly via one or more electronic elements.

1. A device for producing short current pulses with an overall duration of less than one millisecond, said current pulses flowing through at least one coil such that the at least one coil produces magnetic field pulses which cause electric currents in body tissue according to the principle of electromagnetic induction, said electric currents triggering at least one action potential of neurons and/or myocytes by stimulation, wherein the at least one coil is embodied in such a way that a magnetic field generated thereby is able to penetrate the body tissue; wherein the device contains at least one capacitor for storing some or all of the energy required for the magnetic field pulses; characterized in that, for the purposes of reducing the acoustic sound emitted on account of the current pulse by the coil and/or by at least one electric connection cable to the at least one coil, the device is embodied in such a way that at least one electric conductor of the at least one coil and/or the at least one electric connection cable forms a stiff unit by being embedded in a mechanically stiff polymer and/or a mechanically stiff plastic and/or a mechanically stiff composite and/or a mechanically stiff ceramic and/or a mechanically stiff glass.

2. The device as claimed in claim 1, wherein the at least one coil and/or the at least one electric connection cable further contains at least one viscoelastic material layer and/or at least one elastic material layer.

3. The device as claimed in one of claims 1-2, wherein at least one conductor of the at least one coil and/or of the at least one electric connection cable comprises at least two different metals, which may also be different alloys in each case, wherein the at least two metals have at least one interface at which the at least two metals are securely connected to one another mechanically, wherein at least one of the at least two metals has an electrical conductivity which is at least twice as high as that of at least one other metal of the at least two metals, and, at the same time, a Young's modulus which is at most half the size of said at least one other metal of the at least two metals.

4. The device as claimed in one of claims 1-3, wherein the Young's modulus of the at least one elastic material layer is less than one eighth of the Young's modulus of the mechanically stiff polymer and/or of the mechanically stiff plastic and/or of the mechanically stiff composite and/or of the mechanically stiff ceramic and/or of the mechanically stiff glass; and/or wherein either the product's viscosity and Young's modulus of the at least one viscoelastic material layer exceeds 10 billion pascal-squared seconds or the normalized loss factor of the viscoelastic material of the at least one viscoelastic material layer exceeds 0.75.

5. The device as claimed in one of claims 1-4, wherein at least one viscoelastic material layer covers at least one third of the surface of the stiff unit which is formed by the at least one conductor embedded in a mechanically stiff polymer and/or a mechanically stiff plastic and/or a mechanically stiff composite and/or a mechanically stiff ceramic and/or a mechanically stiff glass, and which is connected to this surface in a mechanically adhesive manner, wherein the viscoelastic material layer may be surrounded by further other material layers.

6. The device as claimed in one of claims 1-5, wherein at least one elastic material layer covers at least one third of the surface of the stiff unit, which is formed by the at least one conductor embedded in a mechanically stiff polymer and/or a mechanically stiff plastic and/or a mechanically stiff composite and/or a mechanically stiff ceramic and/or a mechanically stiff glass, and/or at least one viscoelastic material layer partly covering the aforementioned stiff unit.

7. The device as claimed in one of claims 1-6, wherein less than one quarter of the energy of the electric current pulse lies in the frequency range from 500 Hz to 8000 Hz.

8. The device as claimed in one of claims 1-7, wherein the fundamental frequency and/or the dominant frequency of the electric current pulse lies higher than the human hearing limit of 18 kHz.

9. The device as claimed in claim 8, wherein the electric current pulse contains exactly one zero crossing, where the current changes from one polarity to the other, and wherein the overall duration of the current pulse does not exceed 75 microseconds.

10. The device as claimed in claim 8, wherein the electric current pulse comprises a sinusoidal oscillation, the amplitude envelope of which increases from less than one fifth of the maximum to a maximum in less than 500 microseconds and subsequently drops back down to less than one fifth of the maximum in less than 500 microseconds, wherein the frequency of the sinusoidal oscillation may change continuously during the current pulse.

11. A device for producing short current pulses with an overall duration of less than one millisecond in at least one coil such that the at least one coil produces magnetic field pulses with a magnetic flux density of 0.1 to 10 Tesla, said magnetic field pulses causing electric currents in the body tissue according to the principle of electromagnetic induction, said electric currents triggering at least one action potential of neurons and/or myocytes by a stimulus, wherein the at least one coil is embodied in such a way that a magnetic field produced thereby is able to penetrate the body tissue; wherein the device contains at least one capacitor for storing some or all of the energy required for the magnetic field pulses, wherein the stimulating electric currents caused by the magnetic field of the coil are at at least one tenth of and at most ten times the stimulation currents required for stimulating the cells; characterized
in that, for the purposes of reducing the acoustic sound emitted on account of the current pulse by the coil and/or by at least one electric connection cable to the at least one coil, the device is embodied in such a way that at least one electric conductor of the at least one coil and/or the at least one electric connection cable forms a stiff unit by being embedded in a mechanically stiff polymer and/or a mechanically stiff plastic and/or a mechanically stiff composite and/or a mechanically stiff ceramic and/or a mechanically stiff glass; and wherein less than one quarter of the energy of the electric current pulse lies in the frequency range from 500 Hz to 18 kHz.

12. The device as claimed in claim 11, wherein the reduction in the emitted acoustic sound consists of a reduction in the psychoacoustic loudness and/or the peak acoustic sound level and/or the acoustic sound energy and/or the psychoacoustic roughness and/or the psychoacoustic sharpness.

13. The device as claimed in one of claims 11-12, wherein the at least one coil and/or the at least one electric connection cable further contain at least one viscoelastic material layer and/or at least one elastic material layer.

14. The device as claimed in one of claims 11-13, wherein at least one conductor of the at least one coil and/or of the at least one electric connection cable comprises at least two different metals, which may also be different alloys in each case, wherein the at least two metals have at least one interface at which the at least two metals are securely connected to one another mechanically, wherein at least one of the at least two metals has an electrical conductivity which is at least twice as high as that of at least one other metal of the at least two metals, and, at the same time, a Young's modulus which is at most half the size of said at least one other metal of the at least two metals.

15. The device as claimed in one of claims 1-14, wherein the Young's modulus of the at least one elastic material layer is less than one eighth of the Young's modulus of the mechanically stiff polymer and/or of the mechanically stiff plastic and/or of the mechanically stiff composite and/or of the mechanically stiff ceramic and/or of the mechanically stiff glass; and/or wherein the product's viscosity and Young's modulus of the at least one viscoelastic material layer exceeds 10 billion pascal-squared seconds.

16. The device as claimed in one of claims 11-15, wherein at least one viscoelastic material layer covers at least one third of the surface of the stiff unit which is formed by the at least one conductor embedded in a mechanically stiff polymer and/or a mechanically stiff plastic and/or a mechanically stiff composite and/or a mechanically stiff ceramic and/or a mechanically stiff glass, and which is connected to this surface in a mechanically adhesive manner, wherein the viscoelastic material layer may be surrounded by further other material layers.

17. The device as claimed in one of claims 11-16, wherein at least one elastic material layer covers at least one third of the surface of the stiff unit, which is formed by the at least one conductor embedded in a mechanically stiff polymer and/or a mechanically stiff plastic and/or a mechanically stiff composite and/or a mechanically stiff ceramic and/or a mechanically stiff glass, and/or at least one viscoelastic material layer partly covering the aforementioned stiff unit.

18. The device as claimed in one of claims 1-17, wherein the electric current pulse is produced by an electric pulse source containing a multi-level converter with at least three capacitors and producing a current pulse by dynamic electric combination of the at least three capacitors, wherein the electric pulse source can produce current pulses with different amplitude and form, wherein the amplitude and the form may be changed independently of one another between the production of two successive current pulses.

19. The device as claimed in one of claims 1-17, wherein the electric current pulse is produced by an electric pulse source which at least one capacitor (1901, 1902, 2001, 2002) and at least two electronic switches (1903, 1904, 2003, 2004) which are connected electrically in series and which can be switched off, wherein the electric connection between the two electronic switches which can be switched off is connected by way of at least one third electric connection to at least one connector of the coil, either directly or indirectly via one or more electronic elements.
